(19) United States
(12) Patent Application Publication
(10) Pub. No.: US 2025/0170353 A1
(43) Pub. Date: May 29, 2025

(54) FACE MASKS WITH ORAL ATTACHMENT MECHANISMS AND METHODS FOR MAKING THE SAME

(71) Applicant: Doctor's Choice Custom CPAP Mask, LLC, Brea, CA (US)

(72) Inventors: Warren James Schlott, Brea, CA (US); Bryce Nebeker, Brea, CA (US)

(21) Appl. No.: 18/961,231

(22) Filed: Nov. 26, 2024

Related U.S. Application Data
(60) Provisional application No. 63/602,877, filed on Nov. 27, 2023.

(51) Int. Cl. A61M 16/06; A61M 39/12; B33Y 50/00; B33Y 80/00

(52) U.S. Cl. CPC A61M 16/0616; A61M 39/12; B33Y 50/00; B33Y 80/00

(57) ABSTRACT

A face mask assembly may include an oral appliance connected to a post. A face mask assembly may include a face mask comprising a shell, a liner, an adapter, and a threaded, slotted sleeve configured to receive the post. A face mask assembly may include a threaded nut configured to selectively engage the threaded, slotted sleeve, wherein engaging the threaded nut on the threaded, slotted sleeve causes the threaded, slotted sleeve to engage the post and fix the position of the face mask relative to the oral appliance.

### INCORPORATION BY REFERENCE TO ANY PRIORITY APPLICATIONS

The present application claims the benefit of priority to U.S. Ser. No. 63/602,877, filed Nov. 27, 2023, which is incorporated by reference.

### BACKGROUND

This disclosure relates generally to medical devices, and more particularly to a face mask for use in medical and other healthcare applications and method of forming the same.

### SUMMARY

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments will become readily apparent to those skilled in the art from the following detailed description of the preferred embodiments having reference to the attached figures, the invention not being limited to any particular preferred embodiment(s) disclosed.

In one aspect, a face mask assembly is described. The face mask assembly includes: an oral appliance comprising a post, a face mask comprising a shell, a liner, an adapter, and a threaded, slotted sleeve configured to receive the post, and a threaded nut configured to selectively engage the threaded, slotted sleeve, wherein engaging the threaded nut on the threaded, slotted sleeve causes the threaded, slotted sleeve to engage the post and fix the position of the face mask relative to the oral appliance.

In some embodiments, the face mask further comprises an adapter configured to connect with a tube for delivery of a flow of gas to a wearer of the face mask. In some embodiments, the oral appliance is configured to engage the teeth of a wearer. In some embodiments, the oral appliance comprises a lower arch. In some embodiments, the oral appliance comprises an upper arch. In some embodiments, the oral appliance comprises a pad. In some embodiments, the oral appliance comprises a dental grip. In some embodiments, the oral appliance is customized to the mouth of the wearer. In some embodiments, the face mask is customized to the face of a wearer.

In another aspect, a face mask assembly is described. The face mask assembly comprises an oral appliance comprising a post, a face mask comprising a shell, and a releasable attachment mechanism configured to selectively couple the oral appliance and the face mask.

In some embodiments, the releasable attachment mechanism comprises a post sleeve disposed through the shell. In some embodiments, the releasable attachment mechanism further comprises threads disposed on the post and a threaded nut configured to engage the threads disposed on the post. In some embodiments, the releasable attachment mechanism further comprises a toggle lever rotatably attached to the post sleeve, wherein the toggle lever is configured to engage the post and fix the position of the face mask relative to the oral appliance. In some embodiments, the face mask further comprises an adapter configured to connect with a tube for delivery of a flow of gas to a wearer of the face mask. In some embodiments, the oral appliance is customized to the mouth of a wearer. In some embodiments, the face mask is customized to the face of a wearer. In some embodiments, the face mask is sized and shaped to cover at least a portion of the wearer's face, create an airtight seal about features of the wearer's face, and create an air pocket anterior to the wearer's nose and mouth.

In another aspect, a method of making a face mask assembly for a wearer is described. The method includes: scanning the wearer's face, creating a dataset representing the shape of the wearer's face, using the dataset to modify a computer model of a face mask, 3D printing the modified computer model of the face mask to create a face mask, affixing a liner to the face mask, and combining the face mask with an oral appliance and a releasable attachment mechanism configured to selectively couple the oral appliance and the face mask.

In some embodiments, the method further comprises preparing the oral appliance by taking a dental mold of the wearers, and preparing an oral appliance customized to the wearer using the dental mold.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A is a perspective view of an example embodiment of a face mask assembly comprising a face mask secured to its wearer by an oral appliance using a releasable attachment mechanism.

FIG. 1B is an exploded side view of the example embodiment of FIG. 1A.

FIG. 1C is a side view of the face mask of FIG. 1A.

FIG. 1D is a front view of the face mask of FIG. 1A.

FIG. 2 is an exploded side view of an example embodiment of a face mask assembly comprising a face mask secured to its wearer by an oral appliance using a releasable attachment mechanism.

FIG. 3 is an exploded side view of an example embodiment of a face mask assembly comprising a face mask secured to its wearer by an oral appliance using a releasable attachment mechanism.

FIG. 4 is a flowchart illustrating an example method for making a customized face mask assembly.

### DETAILED DESCRIPTION

The foregoing and other aspects of the present disclosure will now be described in more detail with respect to the description and methodologies provided herein. This description is not intended to be a detailed catalogue of all the ways in which the embodiments of the present disclosure may be implemented, or of all the features that may be added to the present disclosure. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant disclosure, will be apparent to those skilled in the art in light of the instant detailed description, figures and claims. Hence, the following specification is intended to illustrate some particular embodiments, and not to exhaustively specify all permutations, combinations and variations thereof.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Face masks are commonly used in many industries to protect the wearers from environmental hazards. Healthcare workers often wear surgical masks or respirators to protect themselves from infectious agents, such as viruses and bacteria. Construction workers may use respirators to safeguard against dust, fumes, and other airborne particulates generated from various construction materials. Workers in manufacturing plants may need respiratory protection when dealing with chemicals, solvents, and aerosolized particles that can be harmful if inhaled. Miners encounter dust and gases like coal dust, silica, and methane, so they use specialized respirators for protection. Farmers and agricultural workers may use masks to shield themselves from pesticides, herbicides, and other agricultural chemicals. Laboratory personnel wear masks when handling hazardous chemicals, toxins, or biological agents.

Other masks, such as the surgical masks worn during the COVID-19 pandemic, may be used to protect people in the wearer's environment from contaminants or infectious agents originating from the wearer. Workers in computer chip manufacturing often wear masks to prevent their breath from releasing particles that could settle on sensitive components.

Other masks are used to deliver a continuous stream of gas to the wearer's airway. Fighter pilots wear oxygen masks to secure a continuous supply of clean oxygen and protect against hypoxia during flight. Face masks are worn by patients undergoing medical procedures so that the medical team can reliably and continually provide an anesthetic to ease pain and calm the patient.

Continuous Positive Airway Pressure (CPAP) masks are used in several applications, including treating a medical condition known as obstructive sleep apnea, a sleep disorder characterized by repeated interruptions in a person's breathing during sleep, minimizing snoring, and others. By delivering a continuous stream of pressurized air, a CPAP system helps to keep the wearer's airways open by preventing the collapse of the throat muscles and tissues. This constant flow of air creates positive pressure, which acts as a splint to hold the airway open, allowing uninterrupted and regular breathing. A typical CPAP patient will wear a CPAP mask during sleep.

Many face masks, including CPAP masks, work best when the masks create an airtight seal around the entry points to the wearer's airways—the nose and mouth. This seal is made by the edges of the mask pressing into the wearer's skin from the bridge of the nose, across both the wearer's cheeks, then on or below the wearer's chin. However, the shape of a person's face can vary widely between individuals. A face mask design that can form a comfortable seal around one wearer's face may leave large gaps around another person's face. For instance, a face mask designed for individuals with high nose bridges may struggle to create an effective seal for those with lower nose bridges. Tightening an ill-fitting mask to create a seal where there is a gap between the mask and the wearer can create uncomfortable pressure around the other points of mask contact.

The seal made by the edges of the mask pressing into the wearer's skin of the face requires some force, both to secure the mask in place and to press it into the skin. Current CPAP masks are secured to a wearer's face using straps that stretch from the edges of the mask around the wearer's head or around the wearer's ears. The straps may be elastic or have a mechanism to adjust their length such that the straps pull the mask toward the back of the wearer's head and into the wearer's face. However, these straps can produce uncomfortable pressure around the back of the wearer's head where the straps dig into the skin of the scalp or pull uncomfortably on the back of the wearer's ears. These straps may slide off too easily or become uncomfortably caught in the wearer's hair. As CPAP masks are generally worn while the wearer sleeps, straps around the back of the head may make it uncomfortable for the wearer to lay down in some positions, and the discomfort caused by the mask and head straps may make it difficult to sleep comfortably.

Thus, face masks that solve these problems are desired. The present disclosure discloses, in part, a customized face mask that is secured to the wearer by a releasable attachment to a customized oral appliance. The customized oral appliance is made to fit in the mask wearer's mouth and be gripped by the wearer's teeth. The customized face mask fits over the wearer's face and connects to the customized oral appliance via a releasable attachment mechanism. The grip of the wearer's teeth on the oral appliance thus secures the mask to the skin of the wearer's face for an airtight seal without the need for uncomfortable straps around the back of the head. The releasable attachment mechanism allows the oral appliance to be placed in and removed from the wearer's mouth without the mask attached. Thus, the mask need not bend out of place nor dig uncomfortably into the wearer's face while the oral appliance is placed or removed in the mouth. The customized oral appliance fits the wearer's mouth comfortably, and the customized mask creates an airtight seal without undue pressure about any unusual features of the wearer's face.

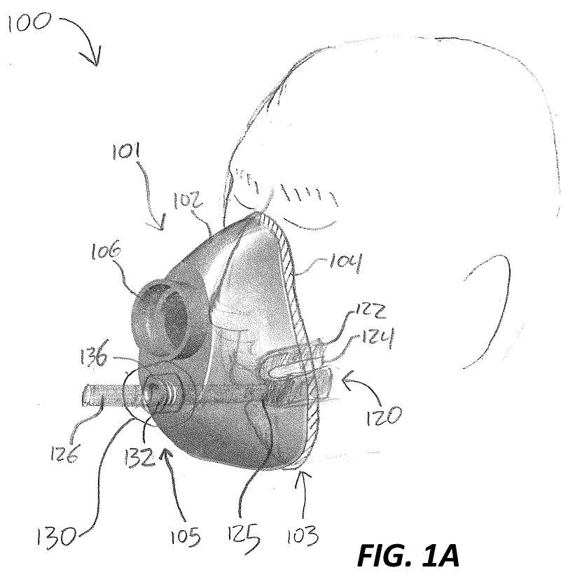

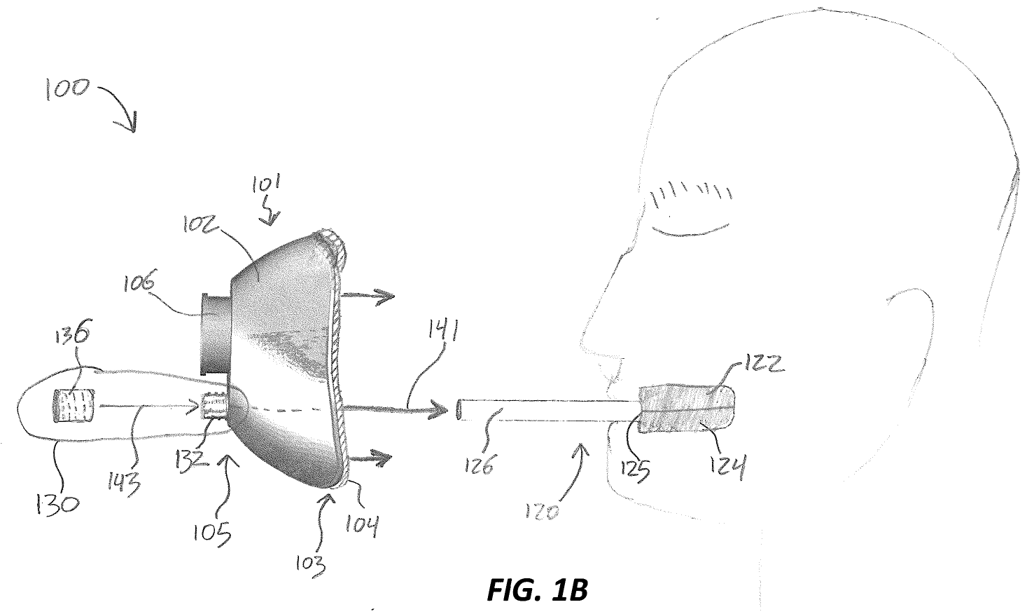

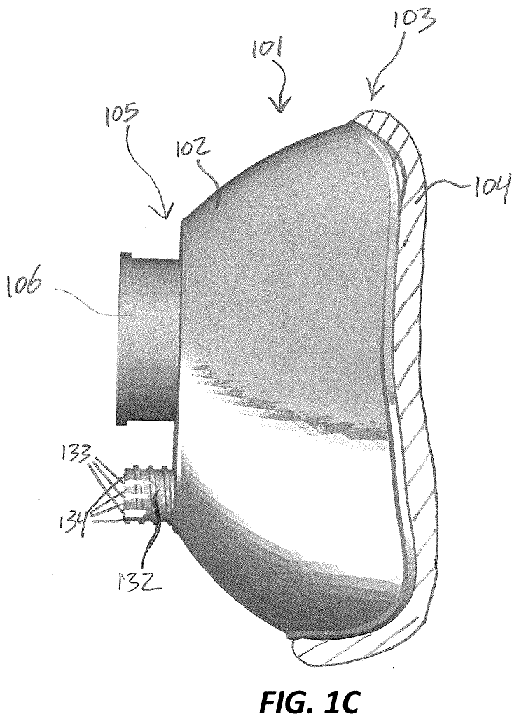

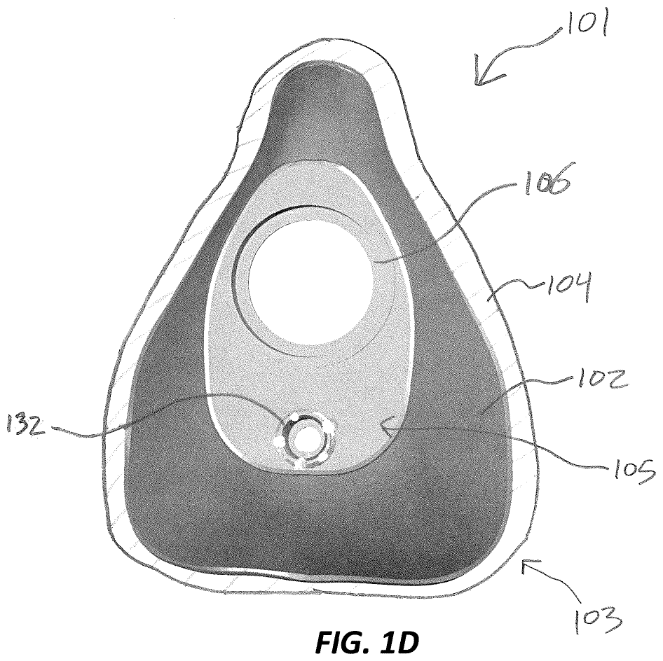

FIG. 1A illustrates an example embodiment of a face mask assembly 100 wherein a face mask is secured to its wearer by an oral appliance using a releasable attachment mechanism. The assembly comprises face mask 101, oral appliance 120, and releasable attachment mechanism 130.

Face mask 101 includes shell 102. Shell 102 is sized and shaped to cover at least a portion of a wearer's face including the mouth and nose of the wearer. A posterior portion 103 of face mask 101 is configured to contact the wearer's face, creating a seal around at least a portion of the wearer's face and an air cavity encompassing the mouth and nose. Face mask 101 also includes liner 104 disposed about at least some portion of the edge of shell 102 such that liner 104 is between shell 102 and the wearer when face mask 101 is in use. In certain embodiments, liner 104 is comprised of a pliable foam or rubber material configured to seal face mask 101 against the wearer's face. Face mask 101 includes adapter 106 configured to connect with a tube for delivery of a flow of gas into face mask 101. Adapter 106 may be disposed on an anterior portion 105 of face mask 101 and comprise a sleeve and hole through shell 102. In certain embodiments, adapter 106 is configured to connect with a CPAP machine via a hose.

Oral appliance 120 is configured to receive the wearer's teeth and position the face mask 101 relative to the wearer. By connecting face mask 101 to the wearer's body, oral appliance 120 maintains face mask 101 in place over the wearer's face and pulls face mask 101 into the skin of the wearer's face for an airtight seal about face mask 101. In this example embodiment, oral appliance 120 includes upper arch 122, lower arch 124, and post 126. Upper arch 122 may be configured to receive a wearer's upper teeth. Lower arch 124 may be configured to receive a wearer's lower teeth. In certain embodiments, oral appliance 120 may be custom-made to fit the dental structure of a particular wearer. For example, a dentist could take an impression of the wearer's dental structure by pressing a putty or a gel or other suitable dental mold making material over the wearer's upper and lower teeth. The dentist may then use the impression of the wearer's upper teeth to form upper arch 122, and the impression of the wearer's lower teeth to form lower arch 124. Because the upper arch 122 and lower arch 124 were formed from the dentist's impressions of the wearer's dental structure, an oral appliance 120 so custom-made may more securely and comfortably receive the wearer's teeth. The more secure and comfortable fit of a custom-made oral appliance 120 may more securely maintain the position and airtight seal of face mask 101 over the wearer's face. In alternate embodiments, oral appliance 120 may be made as a non-custom oral appliance that is sufficiently pliable to form to the wearer's teeth. In alternative embodiments, oral appliance 120 may include post 126 and only one of upper arch 122 and lower arch 124. In some embodiments, oral appliance 120 may include post 126 and a pad such as a rubber pad configured to be gripped by the wearer's teeth.

Post 126 is attached to an anterior end 125 of the oral appliance 120. In certain embodiments, post 126 protrudes from the anterior ends of upper arch 122 and lower arch 124 of oral appliance 120. Post 126 extends past the lips of the wearer when oral appliance 120 is gripped in the wearer's teeth.

The releasable attachment mechanism 130 comprises threaded sleeve 132 and threaded nut 136. Threaded sleeve 132 is disposed on an anterior portion of face mask 101 and extends through shell 102. Threaded sleeve 132 may be threaded on the outside of the sleeve. In certain embodiments, threaded sleeve 132 may comprise slots circumferentially disposed about the sleeve such that it is a threaded, slotted sleeve. The slots may be disposed parallel to a longitudinal axis of threaded sleeve 132, starting at the anterior end of threaded sleeve 132 and at least partially through to the posterior end of threaded sleeve 132. Thus, the anterior end of threaded sleeve 132 may be divided into distinct fins and the diameter of the anterior end of threaded sleeve 132 can be reduced by a squeezing force on threaded sleeve 132. Threaded nut 136 is sized and shaped to apply a squeezing force on threaded sleeve 132 when threaded nut 136 is threaded on threaded sleeve 132.

Post 126 of oral appliance 120 and threaded sleeve 132 disposed on face mask 101 are sized and shaped such that threaded sleeve 132 receives post 126. When oral appliance 120 is received by the wearer's teeth, post 126 is configured such that the distance of face mask 101 from the wearer can be adjusted by sliding face mask 101 along post 126 toward the anterior (away from oral appliance 120 and the wearer's face) or posterior (toward oral appliance 120 and the wearer's face) ends of post 126. By this mechanism, face mask 101 can be placed closer to the posterior end of post 126, and thereby closer to oral appliance 120 and the wearer's face, to form an airtight seal about the wearer's face while the face mask assembly 100 is in use. To remove face mask 101, face mask 101 can be slid along post 126 toward the anterior end of post 126, away from the wearer's face. Thus, face mask 101 can be placed farther from the posterior end of post 126 and the wearer's face, or be slid off the posterior end of post 126 and be removed completely, while placing or removing the oral appliance from the wearer's mouth in anticipation or in termination of use of face mask assembly 100.

Placement and removal of an oral appliance such as oral appliance 120 may involve shifting and angling the oral appliance as it passes the wearer's lips and is received or released by the wearer's teeth. If, during placement and removal of oral appliance 120 from the wearer's mouth, face mask 101 were held at a fixed position relative to oral appliance 120 sufficient to form an airtight seal about the wearer's face during use of the mask, the shifting and angling of oral appliance 120 would deform face mask 101 or uncomfortably press face mask 101 into the skin of the wearer's face while putting on the face mask 101. By means of the innovative face mask assembly 100 described herein, the face mask can be distanced or removed from oral appliance to create room to maneuver oral appliance during placement and removal of oral appliance from the wearer's mouth. This room to maneuver may allow oral appliance 120 to be placed and removed from the wearer's mouth without face mask 101 deforming out of shape or uncomfortably pressing into the skin of the wearer's face. Then face mask can be moved toward the posterior end of post, closer to oral appliance and the wearer's face, and form an airtight seal about the wearer's face while face mask is in use.

Releasable attachment mechanism 130 also comprises threaded nut 136, which is configured to engage threaded sleeve 132. As threaded nut 136 engages threaded sleeve 132, threaded nut 136 applies a squeezing force to fins formed by slots disposed along the anterior end of threaded sleeve 132. These fins, when squeezed, create friction against post 126 and engages post 126 when post 126 is disposed within threaded sleeve 132. When threaded nut 136 is tightened on threaded sleeve 132, threaded nut 136 forces threaded sleeve 132 to engage post 126, fixing the position of face mask 101 relative to oral appliance 120. When loosened, threaded nut 136 allows threaded sleeve 132 to disengage post 126 and allows face mask 101 to slide toward the anterior or posterior ends of post 126.

In certain embodiments, threaded nut 136 is bored completely through and the internal diameter of threaded nut 136 is sufficiently large that post 126 may pass through threaded nut 136. In certain embodiments, threaded nut 136 comprises a ridged exterior for easier grip. In alternative embodiments, threaded nut 136 comprises exterior fins projecting radially for additional torque when tightening and loosening threaded nut 136. In certain embodiments, threaded nut 136 comprises a polymer insert to add resistance against inadvertent loosening.

The releasable attachment mechanism 130 herein described is exemplary, and other releasable attachment mechanisms are possible in accordance with this disclosure.

FIG. 1B illustrates an exploded side view of an example embodiment of face mask assembly 100, which comprises a face mask 101 secured to its wearer by an oral appliance 120 using releasable attachment mechanism 130. FIG. 1B depicts oral appliance 120 in the mouth of the wearer. Upper arch 122 and lower arch 124 of oral appliance 120 receive the wearer's upper and lower teeth, respectively. Oral appliance 120 comprises post 126 which extends from the anterior ends of upper arch 122 and lower arch 124 past the lips of the wearer. Face mask 101 comprises shell 102, liner 104, adapter 106, and threaded sleeve 132. Shell 102 is configured to cover at least a portion of the wearer's face including the mouth and nose of the wearer. Shell 102 may have been custom-made to the contours of the wearer's face, for example by scanning the face and then 3-D printing the shell. A customized mask created by scanning the wearer will fit more perfectly to the contours of the wearer's face. By having a better fit, the disclosed embodiments can provide a mask that has an effective seal about the wearer's face. The disclosed embodiments can be more comfortable because, by being shaped to the user's face, the mask may more evenly distribute pressure around the edges of the mask and not require a strong force against the skin to maintain an effective seal about the edges of the mask.

Threaded sleeve 132 is disposed on and through an anterior portion of shell 102. Threaded sleeve 132 is configured to receive post 126 as within the threaded sleeve 132 face mask 101 is moved toward the anterior, i.e., toward the wearer. In some embodiments, the threads of threaded sleeve 132 are disposed on the exterior of threaded sleeve 132. When correctly positioned along post 126, face mask 101 is configured to form a seal about a portion of the wearer's face through contact between liner 104 and the wearer's skin of the face. Threaded nut 136 is configured to engage and disengage threaded sleeve 132, thereby selectively tightening and loosening the grip of threaded sleeve 132 on post 126. Face mask 101 may include adapter 106 configured to receive a tube providing gas flow into face mask 101. For example, adapter 106 may be configured to receive the end of a hose connected to a CPAP machine such that pressurized air is provided to the wearer through face mask 101.

The example embodiment of face mask assembly 100 in FIGS. 1A and 1B has great advantages over traditional face masks. The wearer's toothy grip on oral appliance 120 discourages up-and-down or side-to-side motion of post 126 relative to the wearer, and thereby up-and-down or side-to-side motion of face mask 101 to which oral appliance 120 is fixed. Threaded nut 136 secures the position of face mask 101 along post 126 so to maintain face mask 101 comfortably on the wearer and maintain a tight seal about the wearer's face. By loosening threaded nut 136, repositioning face mask 101 along to post 126 and relative to the wearer, then tightening threaded nut 136, the force pressing face mask 101 into the face of wearer can be adjusted. Such adjustments can help find the correct balance between competing needs for a tight seal about the face and a comfortable fit for the wearer. The disclosed embodiments may lack straps that can cause discomfort to the wearer's head or ears, instead securing the mask by means of the user's teeth.

To put face mask assembly 100 in use in covering at least a portion of the face of a wearer, oral appliance 120 is separated from face mask 101 and placed in the mouth of the wearer. Oral appliance 120 is positioned in the mouth such that upper arch 122 receives the wearer's upper teeth, lower arch 124 receives the wearer's lower teeth, and post 126 extends past the wearer's lips. Releasable attachment mechanism 130 is adjusted to allow movement of face mask 101 along post 126 by at least partially disengaging threaded nut 136. Arrow 141 shows where threaded sleeve 132 is slid onto the anterior end 127 of post 126 (distal from anterior end 125 of oral appliance 120). Threaded sleeve 132 is then slid along post 126 toward anterior end 125 of oral appliance oral appliance 120 while maintaining the position of oral appliance 120 in the mouth of the wearer, thus moving face mask 101 into position over the face of the wearer. When anterior portion 105 of face mask 101 is in position to cover and receive at least a part the wearer's face and form a seal about the skin of the wearer's face, releasable attachment mechanism 130 is engaged to secure the position of face mask 101 relative to oral appliance 120 and the wearer's face. Here, releasable attachment mechanism 130 is engaged when threaded nut 136 is engaged on threaded sleeve 132. Arrow 143 shows where threaded nut 136 is engaged on threaded sleeve 132. When threaded nut 136 is engaged on threaded sleeve 132, the squeezing force on threaded sleeve 132 creates friction between threaded sleeve 132 and post 126.

To remove face mask assembly 100, the actions are reversed. First, threaded nut 136 is disengaged from threaded sleeve 132 to allow threaded sleeve 132 to slide freely over post 126. Then face mask 101 is moved toward the anterior, away from the face of the wearer as threaded sleeve 132 is slid along post 126 away from oral appliance 120. Face mask 101 is slid off anterior end 127 end of post 126 and removed. Then, oral appliance 120 is removed from the mouth of the wearer.

For small adjustments of face mask assembly 100 while in use, releasable attachment mechanism 130 may be partially disengaged to allow threaded sleeve 132 to slide in small increments over post 126. The wearer can make adjustments on face mask assembly 100 by engaging or disengaging releasable attachment mechanism 130 using finger strength.

FIG. 1C illustrates a side view of an example embodiment of face mask 101 and threaded sleeve 132 of releasable attachment mechanism 130. As described above, face mask 101 comprises shell 102 and liner 104, with a posterior portion 103 of face mask 101 shaped and sized to receive at least a portion of a wearer's face. Disposed on an anterior portion 105 of face mask 101 is adapter 106 configured to connect with a tube for delivery of a flow of gas into face mask 101.

Also disposed on an anterior portion 105 of face mask 101 is threaded sleeve 132 of releasable attachment mechanism 130. In this example embodiment, threaded sleeve 132 is externally threaded. Threaded sleeve 132 comprises a plurality of slots 133 disposed along at least a portion of the anterior end of threaded sleeve 132. These plurality of slots 133 divide the anterior end of threaded sleeve 132 into a plurality of fins 134. The plurality of slots 133 compress when a threaded nut 136 is engaged on threaded sleeve 132, and the plurality of fins 134, squeezed by threaded nut 136, then create friction against post 126 of oral appliance 120 and engage post 126.

FIG. 1D illustrates a front (anterior) view of an example embodiment of face mask 101 and threaded sleeve 132 of releasable attachment mechanism 130. As described above, face mask 101 comprises shell 102 and liner 104, with a posterior portion 103 of face mask 101 shaped and sized to receive at least a portion of a wearer's face. Disposed on an anterior portion 105 of face mask 101 is adapter 106 configured to connect with a tube for delivery of a flow of gas into face mask 101. Adapter 106 includes a cavity in shell 102 to allow gas to pass into face mask 101. Threaded sleeve 132 includes a cavity in shell 102 to allow post 126 to pass through face mask 101 from oral appliance 120 to threaded sleeve 132 where it can be engaged by releasable attachment mechanism 130.

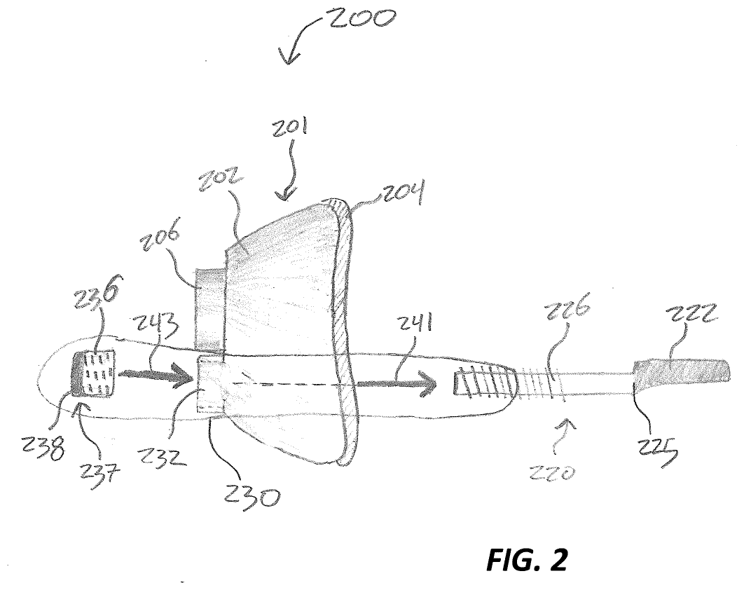

FIG. 2 illustrates an exploded side view of an example embodiment of a face mask assembly 200 comprising a face mask secured to its wearer by an oral appliance. Face mask assembly 200 includes face mask 201, oral appliance 220, and releasable attachment mechanism 230. Features of face mask assembly 200 can be similar to the features of other face mask assemblies described herein.

Oral appliance 220 is comprised of a dental grip 222 and a threaded post 226. Dental grip 222 is configured to be placed in the mouth of a wearer and gripped by the wearer's teeth. In certain embodiments, dental grip 222 is comprised of a rubbery substance such that dental grip 222 is not easily torn by the strength of a bite and the wearer's teeth. In certain embodiments, dental grip 222 is configured to receive the wearer's teeth, such as by having an indent in the surface of dental grip 222 to receive the upper teeth. In certain embodiments, dental grip 222 is formed based on a dental mold of the wearer's teeth such that the top and bottom surface of dental grip 222 show the wearer's teeth in relief and seat the wearer's teeth when in use. Threaded post 226 projects toward the anterior from an anterior end 225 of dental grip 222. Threaded post 226 is configured to be of sufficient length that it projects past the wearer's lips when oral appliance 220 is placed in use and dental grip 222 is properly seated between the wearer's teeth.

Features of face mask 201 can be similar to features of face masks described elsewhere herein. Face mask 201 comprises of shell 202, liner 204, and adapter 206. Shell 202 is configured to cover at least a portion of the face of a wearer including the wearer's nose and mouth and create an air pocket anterior to the nose and mouth. Liner 204 is disposed along at least part of a posterior edge of shell 202. Liner 204 and shell 202 are configured such that the posterior edge of face mask 201 aligns with the contours of the face and creates a seal. In certain embodiments, shell 202 is created custom for a particular wearer. In certain embodiments, the wearer's face is scanned, creating a dataset representing the shape of at least a part of the wearer's face. In certain embodiments, a custom shell 202 is custom manufactured, such as by 3D printing. In certain embodiments, the custom manufacturing may use at least partially the dataset created from the scan to map and print a posterior edge of shell 202 matching the shape of at least a part of the wearer's face. In certain embodiments, face mask 201 includes adapter 206 configured to connect with a tube for delivery of a flow of gas into face mask 201. Adapter 206 may be disposed on an anterior portion of face mask 201 and comprise a sleeve and hole through shell 202. In certain embodiments, adapter 206 is configured to connect with a CPAP machine via a hose.

Releasable attachment mechanism 230 comprises threaded nut 236, post sleeve 232, and threaded post 226 of oral appliance 220. Post sleeve 232 is disposed on an anterior portion of face mask 201 and through shell 202. In certain embodiments, post sleeve 232 may project to the anterior of the main body of face mask 201. Post sleeve 232 is configured to receive, but not engage threaded post 226 such that face mask 201 may freely slide toward the anterior or posterior along threaded post 226. Threaded nut 236 is internally threaded and configured to engage threaded post 226. In some embodiments, threaded nut 236 may comprise a polymer insert 238 disposed on an anterior portion 237 of threaded nut 236 to add resistance against inadvertent loosening.

When using face mask 201, oral appliance 220 is placed in the wearer's mouth, allowing the wearer's teeth to engage dental grip 222 while threaded post 226 protrudes past the wearer's lips. Face mask 201 is then placed over at least a portion of the wearer's face by moving it toward the posterior of the wearer. In some configurations, post sleeve 232 follows arrow 241 such that post sleeve 232 slides over threaded post 226 until threaded post 226 protrudes through post sleeve 232. Threaded nut 236 is then engaged to threaded post 226 following arrow 243. Post sleeve 232 is configured to not allow threaded nut 236 to pass through face mask 201. Thus, engaging the posterior portion of threaded post 226 that protrudes past post sleeve 232 with threaded nut 236 fixes anterior movement of face mask 201. Face mask 201 is thereby secured relative to the face of the wearer by connection to the wearer's teeth.

In some embodiments, threaded nut 236 comprises a flange disposed radially on an anterior portion 237 of threaded nut 236. The main body of threaded nut 236 is configured to fit within post sleeve 232 and receive and engage oral appliance 220, while the flange prevents threaded nut 236 from passing through post sleeve 232. Threaded nut 236 is then engaged with threaded post 226, securing the position of face mask 201 relative to the face of the wearer.

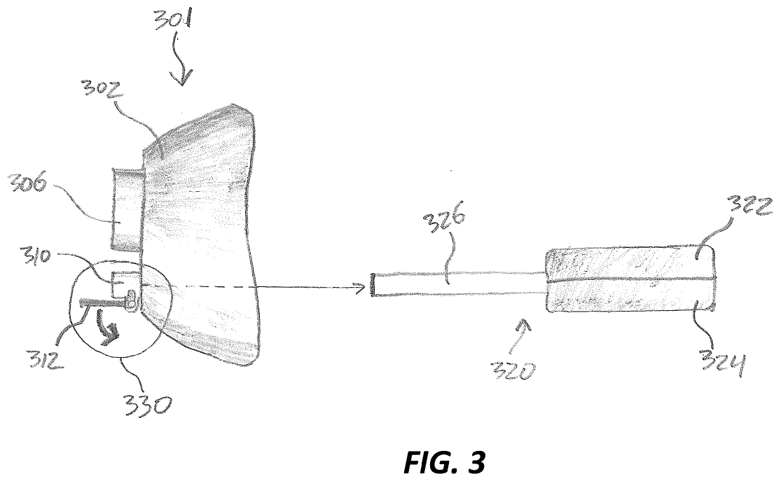

FIG. 3 illustrates an exploded side view of an example embodiment of a face mask assembly 300 secured to its wearer by an oral appliance. Face mask assembly 300 is comprised of face mask 301, oral appliance 320, and releasable attachment mechanism 330.

Oral appliance 320 is comprised of an upper arch 322 configured to receive a wearer's upper teeth, a lower arch 324 configured to receive a wearer's lower teeth and post 326. Post 326 is attached to an anterior portion of upper arch 322 and/or lower arch 324. Post 326 protrudes toward the anterior. Post 326 is configured such that when upper arch 322 and lower arch 324 are engaged by the wearer's teeth, post 326 protrudes past the wearer's lips.

Face mask 301 is comprised of shell 302, and post sleeve 310. Shell 302 is configured to cover at least a portion of the face of a wearer including the wearer's nose and mouth and create an air pocket anterior to the nose and mouth. Shell 302 is configured such that the posterior edge of face mask 301 aligns with the contours of the face and creates a seal. In certain embodiments, shell 302 is created custom for a particular wearer. In certain embodiments, the wearer's face is scanned, creating a dataset representing the shape of at least a part of the wearer's face. In certain embodiments, shell 302 is 3D printed, utilizing at least partially the dataset to map and print a posterior edge of shell 302 matching the shape of at least a part of the wearer's face. In certain embodiments, face mask 301 includes adapter 306 configured to connect with a tube for delivery of a flow of gas into face mask 301. Adapter 306 may be disposed on an anterior portion of face mask 301 and comprise a sleeve and hole through shell 302. In certain embodiments, adapter 306 is configured to connect with a CPAP machine via a hose.

Releasable attachment mechanism 330 is comprised of post sleeve 310 and toggle lever 312. Post sleeve 310 is disposed on an anterior portion of face mask 301 and through shell 302. Post sleeve 310 is configured to receive and selectively retain post 326 by means of a toggle lever 312 rotatably attached to the post sleeve. Toggle lever 312 is configured to rotate about an axis between a closed position and an open position. When in the closed position, a section of toggle lever 312 protrudes into post sleeve 310 and fixes the position of face mask 301 relative to oral appliance 320 via friction with post 326. When in the open position, the portion of toggle lever 312 engaging post 326 withdraws, allowing the position of face mask 301 along post 326 to be adjusted. In other embodiments, toggle lever 312 may incorporate a spring returning toggle lever 312 to the closed position when an outside force is not rotating toggle lever 312 into the open position.

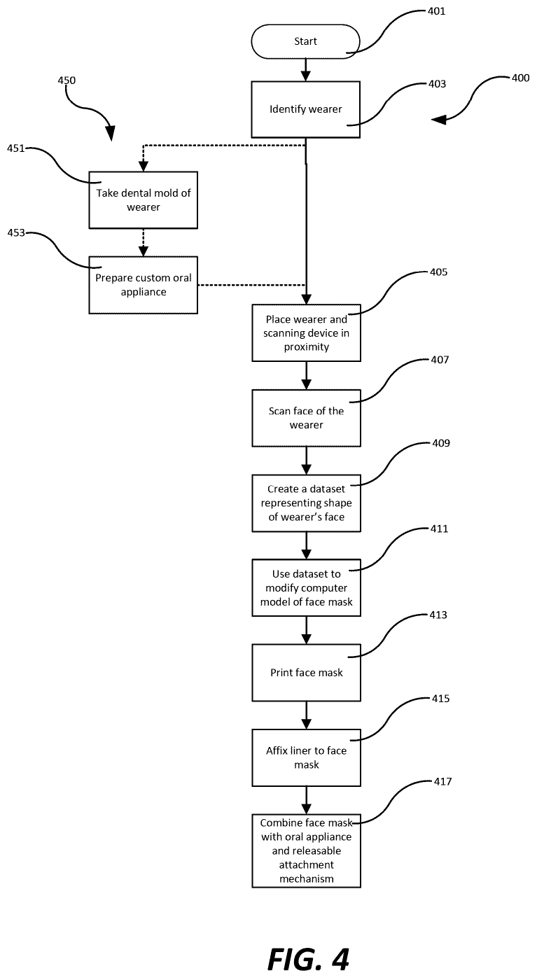

FIG. 4 is a flowchart illustrating an example method 400 of making a face mask assembly as heretofore described. The method 400 begins at a start block 401.

The method then moves to block 403, at which a person needing a face mask is identified. As an example, a person needing a mask may be identified when a physician recommends a patient start on treatment using a CPAP machine, requiring that person to obtain a face mask. That person could then visit the dentist to be fitted for a custom oral appliance, as described in method 450 below. As another example of identifying a person needing a face mask, a chemical laboratory may receive a new hire for training who requires a mask as part of the hire's personal protective equipment. This person needing a mask is an intended mask wearer.

The method then moves to block 405, at which the intended mask wearer is placed in front of a scanning device in communication with a computer such that the wearer and scanning device are properly orientated for the scanning device to scan the intended mask wearer's face.

The method then moves to block 407, where a dentist or technician takes a scan of the intended mask wearer's face using the scanner.

The method then moves to block 409, wherein the scanning device or computer in communication therewith creates a dataset representing at least a portion of the surface of the intended mask wearer's face. The dataset may include a measurement of depth of different parts of the surface of the intended mask wearer's face.

The method then moves to block 411, at which a computer uses the dataset to modify a preset computer model of a face mask to accommodate at least a portion of the surface of the wearer's face. In some embodiments, the preset computer model of the face mask may comprise the anterior parts of a face mask with components of a releasable attachment mechanism and adapter for gas flow. The model of the face mask is then modified to include a posterior portion adapted to the wearer's face as represented in the dataset created in block 409. In some embodiments, the preset computer model of the face mask may comprise the anterior parts of a face mask with components of a releasable attachment mechanism and adapter for gas flow together with a generic posterior portion fitting an average person's face. The model is then modified such that the posterior portion is adapted to the wearer's face as represented in the dataset created in block 409. For example, if the dataset indicates the wearer has a high bridge of the nose, the computer model of a face mask may be modified to create a deeper recess at a portion of the face mask intended to receive and/or seal against the bridge of the wearer's nose.

The method then moves to block 413, at which a face mask is created by additive manufacturing, such as by 3D printing the modified computer model of a face mask. The method then moves to block 415, at which a liner is affixed to face mask. The method then moves to block 417, at which an oral appliance and a releasable attachment mechanism are combined with the face mask to form a face mask assembly.

The example method 400 also comprises method 450 for making a custom oral appliance for combining with face mask and releasable attachment mechanism in block 417. FIG. 4 illustrates method 450 as being performed between blocks 403 and 405. Though FIG. 4 illustrates method 450 as occurring between blocks 403 and 405, it will be understood by those of skill in the art that method 450 could occur anywhere between blocks 403 and 417, e.g., between blocks 405 and 407, between blocks 407 and 409, etc. This is illustrated in FIG. 4 by the use of dotted arrow lines between blocks in method 450 and other blocks in method 400.

After identifying the intended wearer in block 403, a dental mold or impression of the wearer is taken at block 451. For example, a soft putty can be placed in a holder and then pressed against the upper or lower teeth of the wearer to create an impression of the teeth. As another example, a “boil-and-bite” type device that includes a thermoplastic material may be heated and then bitten by the wearer to create a mold. At block 453, a custom oral appliance 120 comprising a post (such as post 126 of FIGS. 1A-1B, threaded post 226 of FIG. 2, or post 326 of FIG. 3) is prepared from the dental mold or impression. For example, a “boil-and-bite” type device may already incorporate a post and require cooling to harden after taking the impression. As another example, a mold can be prepared from the impression taken of the wearer's teeth, where the mold has an upper arch, a lower arch, or both, and then attached to a post. Then later, at block 417, the custom oral appliance may be combined with the customized face mask and a releasable attachment mechanism. In some embodiments of methods of making a face mask assembly, the method does not comprise preparing a customized face mask as shown in blocks 405 through 415. For example, in some embodiments, the method begins at start block 401, then proceeds to block 403 wherein a wearer is identified. The method then proceeds to method 450, comprising block 451 wherein a dental mold of the wearer is taken, and block 453 wherein a custom oral appliance is prepared. The method then proceeds to block 417 wherein the oral appliance is combind with a face mask and releaseable attachment mechanism.

Although the following terms are believed to be well understood by one of skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

As used herein, the terms “a” or “an” or “the” may refer to one or more than one. For example, “a” marker can mean one marker or a plurality of markers.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “and/of” refers to and encompasses any and all possible combinations of one or more of the associated listed items, as well as the lack of combinations when interpreted in the alternative (“of”).

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

As used herein, the term “consists essentially of” (and grammatical variants thereof), as applied to the compositions and methods of the present disclosure, means that the compositions/methods may contain additional components so long as the additional components do not materially alter the composition/method. The term “materially alter,” as applied to a composition/method, refers to an increase or decrease in the effectiveness of the composition/method of at least about 20% or more. For example, a component added to a composition of the present disclosure would “materially alter” the composition if it increases or decreases the composition's ability to inhibit tumor growth by at least 20%.

Those of skill will recognize that the various illustrative logical blocks, modules, circuits, and algorithm steps described in connection with the embodiments disclosed herein may be implemented as electronic hardware, software stored on a computer readable medium and executable by a processor, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

What is claimed is:

1. A face mask assembly, comprising:
an oral appliance comprising a post;
a face mask comprising a shell, a liner, an adapter, and a threaded, slotted sleeve configured to receive the post; and
a threaded nut configured to selectively engage the threaded, slotted sleeve;
wherein engaging the threaded nut on the threaded, slotted sleeve causes the threaded, slotted sleeve to engage the post and fix the position of the face mask relative to the oral appliance.

2. The face mask assembly of claim 1, wherein the face mask further comprises an adapter configured to connect with a tube for delivery of a flow of gas to a wearer of the face mask.

3. The face mask assembly of claim 1, wherein the oral appliance is configured to engage the teeth of a wearer.

4. The face mask assembly of claim 3, wherein the oral appliance comprises a lower arch.

5. The face mask assembly of claim 3, wherein the oral appliance comprises an upper arch.

6. The face mask assembly of claim 3, wherein the oral appliance comprises a pad.

7. The face mask assembly of claim 3, wherein the oral appliance comprises a dental grip.

8. The face mask assembly of claim 3, wherein the oral appliance is customized to the mouth of the wearer.

9. The face mask assembly of claim 1, wherein the face mask is customized to the face of a wearer.

10. A face mask assembly, comprising:
an oral appliance comprising a post;
a face mask comprising a shell; and
a releaseable attachment mechanism configured to selectively couple the oral appliance and the face mask.

11. The face mask assembly of claim 10, wherein the releasable attachment mechanism comprises a post sleeve disposed through the shell.

12. The face mask assembly of claim 11, wherein the releasable attachment mechanism further comprises threads disposed on the post and a threaded nut configured to engage the threads disposed on the post.

13. The face mask assembly of claim 11, wherein the releaseable attachment mechanism further comprises a toggle lever rotatably attached to the post sleeve, wherein the toggle lever is configured to engage the post and fix the position of the face mask relative to the oral appliance.

14. The face mask assembly of claim 10, wherein the face mask further comprises an adapter configured to connect with a tube for delivery of a flow of gas to a wearer of the face mask.

15. The face mask assembly of claim 10, wherein the oral appliance is customized to the mouth of a wearer.

16. The face mask assembly of claim 10, wherein the face mask is customized to the face of a wearer.

17. The face mask assembly of claim 16, wherein the face mask is sized and shaped to cover at least a portion of the wearer's face, create an airtight seal about features of the wearer's face, and create an air pocket anterior to the wearer's nose and mouth.

18. The face mask assembly of claim 17, wherein the face mask further comprises an adapter configured to connect with a tube for delivery of a flow of gas to a wearer of the face mask.

19. A method of making a face mask assembly for a wearer, the method comprising:
scanning the wearer's face;
creating a dataset representing the shape of the wearer's face;
using the dataset to modify a computer model of a face mask;
3D printing the modified computer model of the face mask to create a face mask;
affixing a liner to the face mask; and
combining the face mask with an oral appliance and a releaseable attachment mechanism configured to selectively couple the oral appliance and the face mask.

20. The method of claim 19, wherein the oral appliance is prepared by taking a dental mold of the wearer, and preparing an oral appliance customized to the wearer using the dental mold.
